(19) United States
(12) Patent Application Publication
(10) Pub. No.: US 2010/0203008 A1
(43) Pub. Date: Aug. 12, 2010

(54) NOVEL COMPOSITIONS FOR THE TREATMENT OF VERIOUS INFLAMMATORY CONDITIONS

(75) Inventor: Lars Hellman, Uppsala (SE)

Correspondence Address: BACON & THOMAS, PLLC, 625 SLATERS LANE, FOURTH FLOOR, ALEXANDRIA, VA, 22314-1176, US

(73) Assignee: Theravac Pharmaceuticals AB, Uppsala (SE)

(21) Appl. No.: 12/312,488

(22) PCT Filed: Nov. 12, 2007
(86) PCT No.: PCT/SE2007/000998

§ 371 Date: May 13, 2009

(30) Foreign Application Priority Data
Nov. 13, 2006 (SE) 0602414-5

(51) Int. Cl. A61K 38/20; C07K 14/54; A61P 29/00; A61P 37/08; C12N 15/09

(52) U.S. Cl. 424/85.2; 530/351; 435/69.3

(57) ABSTRACT

The invention relates to compositions for the treatment of inflammatory conditions. The compositions are vaccines comprising IL-18 or fragments thereof.

The present invention relates to methods designed to alleviate the symptoms or prevent the induction of various inflammatory conditions by targeting the cytokine IL-18. Although the invention generally relates to a vaccine for use in a mammal, preferred embodiments thereof relates to vaccines for the use in human, dog, cat or horse, the invention will be described below generally, and with reference to such vaccines for human, feline, equine or canine use.

### BACKGROUND OF THE INVENTION

During the past few decades several diseases caused by the malfunction of the immune system have become major challenges of modern day medicine. Two such areas are the allergic and autoimmune diseases. Allergies have in man become almost epidemic during the past 20-30 years and atopic, or IgE-mediated allergies, are the dominating form. Allergies are also a major problem for many domestic animals like dogs, cats and horses. However, the involvement of IgE is here less well documented. Common types of atopic allergies include in man, fur allergies, hay fever, dust mite allergies, insect venom allergies, extrinsic asthma and many types of food allergies. In addition, many of our domestic animals suffer from allergies directed against similar allergens. Many autoimmune diseases have also increased in frequency and the total number of people affected by various autoimmune diseases is estimated to 3-5%. These two immune related diseases thereby affect almost 25% of the population.

Both allergic and autoimmune diseases are caused by malfunctions in our immune system and have many regulatory mechanisms in common. Several of the cytokines regulating normal immune responses against various pathogens appear also to be directly involved in these disease processes. Cytokines, or growth and differentiation factors of importance for the regulation of our immune system may thereby serve as potential targets for intervention.

One particular difficult problem in veterinary medicine is severe atopic dermatitis in dogs. Almost 50% of all visits to the veterinarians are due to skin problems where atopic dermatitis is the dominating factor. There is a similar situation in human medicine. However here the condition is severe asthma. For the most severe cases non of the existing treatment regiments show sufficient clinical effect. In the situation with severe atopic dermatitis in dogs many of these dogs have to be removed. We have here a great unmet medical need.

We have for many years been working on a potential treatment strategy against IgE mediated allergies in man and domestic animals, vaccination against IgE [1-5]. However in cases where IgE levels are exceptionally high as in dogs [6], this strategy has its clear limitations. These limitations are primarily due to strong tolerizing effects induced by the high concentrations of circulating IgE. In addition, it is difficult to obtain good clinical effects when large amounts of the target molecule have to be removed. We therefore saw it as almost impossible to reach our goal in dogs by vaccinating against IgE [6]. New innovative strategies had to be developed. We here present one potential solution to the problem, vaccination against one important pro-inflammatory cytokine IL-18.

IL-18 is a cytokine, which together with IL-12 has been shown to be a very important inducer of a cell-mediated immunity. The effect on cell-mediated immunity is to a large extent mediated by the induction of interferon-gamma production by NK-cells (natural killer cells) and several other cell types. In addition, IL-18 was recently shown to be an important inducer also of IgE production and to induce symptoms similar to two important cytokines involved in the early events in the induction of allergy, IL4 and IL13. This latter finding indicated that IL-18 also plays a role in the second major arm of adaptive immunity, humoral immune responses. This arm is categorized as a TH2 type of immunity. Overproduction of IL-18 in keratinocytes by introducing a transgenic copy of the gene with a promoter from a keratinocyte specific gene resulted in severe atopic dermatitis in the transgenic mice [7]. Deletion of a signal-transducing gene, STAT6, involved in the transcriptional activation of several genes as a consequence of IL4 signaling did not affect this effect by IL-18 [7]. IL-18 has also been shown to induce asthma like symptoms indicating that it may be involved in several TH2 mediated inflammatory conditions [7]. This makes IL-18 a highly interesting target for novel treatment strategies, particularly against atopic dermatitis and asthma. In addition, due to the potent interferon-gamma inducing effect of IL-18 this cytokine may also have very important effects in many autoimmune disorders making it an interesting target also for these types of inflammatory conditions. I here describe a new way to modulate the IL-18 dependent inflammation, which may become a new important step in the management of severe asthma in humans and atopic dermatitis in dogs.

### THE PRIOR ART

Patent applications describing the use of monoclonal antibodies or soluble receptors for blocking the activity of IL-18 has been filed (WO0158956 A2). These strategies for targeting IL-18 is dependent on injections of highly purified recombinant protein every two to four weeks possibly for the rest of the life of the patient. A vaccine, as described in this application, could here serve as a major improvement over prior art due to that it rely on injections of recombinant protein in a much smaller scale, maybe as little as 1/10 000 compared to the amount needed for treatment with monoclonals or soluble receptor. A vaccines most likely also needs to be administrated one to four times a year as compared to the much more frequent administrations of monoclonals or soluble receptor as described above.

### OBJECT OF THE INVENTION

The object of the invention is to provide a convenient and cost effective method to treat various inflammatory conditions caused by excessive activation by IL-18. Treatment with a vaccine with a fusion protein consisting of IL-18 (or parts of IL-18) and a foreign carrier protein reduces the levels of free IL-18 and thereby reduces the symptoms caused by excessive release of IL-18.

### SUMMARY OF THE INVENTION

The above object is achieved according to the invention by a vaccine, which is characterized by containing a protein having the entire amino acid sequence or IL-18 from the species to be vaccinated or a segment larger than 5 amino acids of said amino acid sequence, in its original or multimerized form. The protein may optionally be coupled to one or more heterologous carrier proteins and by optionally containing an adjuvant.

### BRIEF DESCRIPTION OF THE FIGURES

FIGS. 1 A and B shows the nucleotide and the corresponding amino acid sequence of canine and human IL-18.

FIG. 2 shows a schematic representation of three different constructs made for mouse and dog IL-18 fusion proteins, one with GST from the parasitic worm Schistozoma japonicum as fusion partner and two different variants with the bacterial thioredoxin from E. coli as fusion partner. The figure also shows the purified GST-moue IL-18 fusion protein used for immunization studies.

### DESCRIPTION OF THE INVENTION

Anti-IL-18 antibodies are produced in the host by active immunization, so called vaccination. By injecting a modified IL-18 molecule into the host the immune system of the host produces a polyclonal antibody response directed against its own IL-18 thereby down regulating the effects of its potentially excessive IL-18 production. It is of major importance to modify the antigen so that the immune system of the host recognize the modified self-protein as a non-self protein. This can be achieved by covalent coupling of non-self amino acid regions to IL-18 or a selected region of IL-18 from the species to be treated. The peptides within the non-self region then attract and activate non-tolerized T cells, which give help for the potentially auto-reactive B cells.

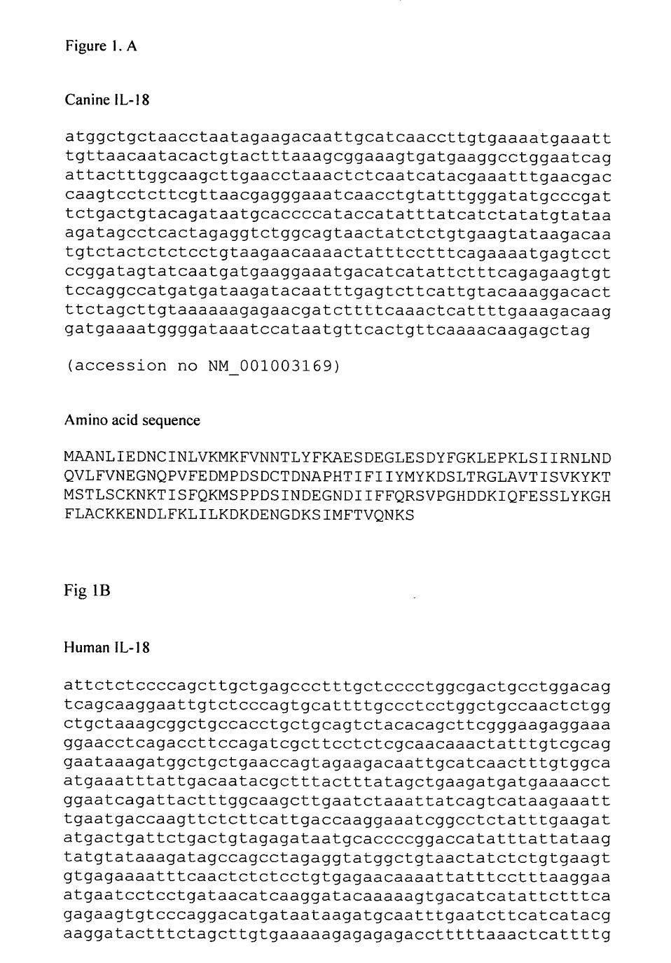

There are at least four possible strategies to do this modification of the self-protein. One method is to produce a fusion protein between a non-self protein, and the entire or a selected fragment of more than 5 amino acids of self IL-18 in a prokaryotic or eukaryotic expression system. The open reading frame of IL-18, as exemplified by canine and human IL-18 in FIG. 1, is then first being cloned into a bacterial, fungal or eukaryotic fusion protein vector. This fusion protein construct is then transfected into a mammalian or prokaryotic host for production of the desired fusion protein. The fusion partner can here be any non-self protein of any size from 10 amino acids to several hundred kD. However, it is usually favorable to use a fusion partner of approximately the same size as the self-protein.

Alternatively, an immunodominant peptide can be inserted into the IL-18 structure giving rise to a fusion protein with self-IL-18 sequences on both sides of the foreign peptide.

As a third alternative, a non-modified IL-18 can be produced in a mammalian or prokaryotic host or host cell line and then covalently attached to a carrier protein by chemical coupling.

The fourth alternative, which in our mind less favorable, is to produce selected regions of the IL-18 sequence as synthetic peptides and then to couple these peptides to a foreign carrier molecule by chemical coupling. This fourth alternative usually results, after injection into the patient, in antibody responses that show low binding activity against the native properly folded protein and thereby in lower clinical effect.

Following production the vaccine antigen is then purified and tested for pyrogen content and potential content of other contaminants. In order to obtain sufficiently strong immune response against the self-epitopes the vaccine antigen is then (optionally) mixed with an adjuvant before injection into the patient. After administration in the patient the vaccine induces an immune response against the vaccine antigen. Due to the presence of self-epitopes in the vaccine antigen this protein also induces an antibody response against the target molecule, here IL-18, thereby reducing the levels of this protein in the patient.

### Example

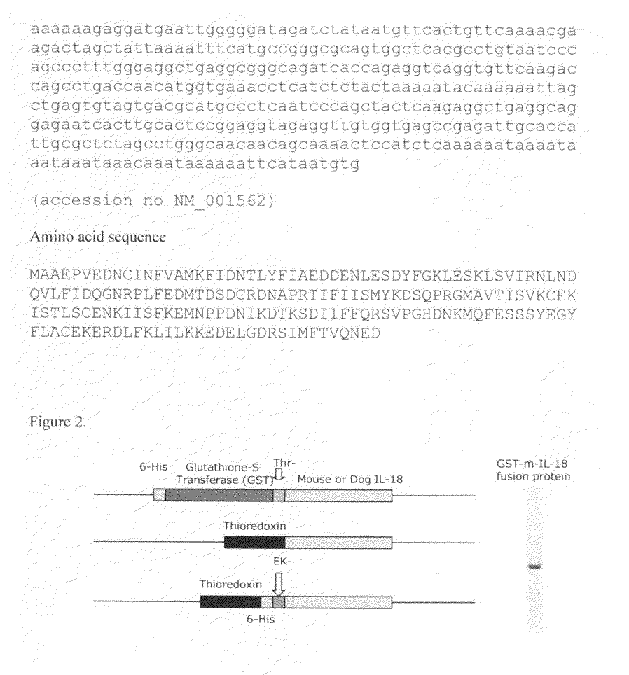

Fusion proteins for both mouse and dog IL-18 have been produced in a prokaryotic host. Three different variants of the vaccine has been constructed, one with the glutathione-S-transpherase (GST) from the parasitic worm Schistosoma japonicum as fusion partner and two variants with a bacterial protein, the thioredoxin from E. coli, as fusion partner (FIG. 2). In order to test for the potency of this fusion protein to induce a therapeutic anti-IL18 response, the mouse GST-IL-18 fusion protein was injected into mice, together with an adjuvant. After three weeks the mice received a booster dose of the vaccine and after five weeks of treatment serum from these mice were tested for the amount of anti-IL-18 antibodies produced. The vaccine induced the production of significant levels of anti-IL-18 in all mice tested. This shows that the vaccine have the capacity to induce the production substantial amounts of anti-IL-18 antibodies in a test animal.

### REFERENCES

1 Hellman, L. Profound reduction in allergen sensitivity following treatment with a novel allergy vaccine. Eur J Immunol 1994, 24(2), 415-420.
2 Hellman, L. Is vaccination against IgE possible? Adv Exp Med Biol 1996, 409, 337-342.
3 Hellman, L. & Carlsson, M. Allergy vaccines: A review of developments. Clin Immunotherapeutics 1996, 6(2), 130-142.
4 Hellman, L. Vaccines against allergies. In Handbook of Experimental Pharmacology, Vol. 133, Vaccines, Vol. 133 (Eds. Perlmann, P. & Wigzell, H.) Springer-Verlag, Berlin, 1999. 499-526.
5 Vernersson, M., Ledin, A., Johansson, J. & Hellman, L. Generation of therapeutic antibody responses against IgE through vaccination. Faseb J 2002, 16(8), 875-877.
6 Ledin, A., Bergvall, K., Salmon-Hillbertz, N. et al. Generation of therapeutic antibody responses against IgE in dogs, an animal species with exceptionally high plasma IgE levels. Vaccine 2006, 24, 66-74.
7 Tsutsui, H., Yoshimoto, T., Hayashi, N., Mizutani, H. & Nakanishi, K. Induction of allergic inflammation by interleukin-18 in experimental animal models. Immunol Rev 2004, 202, 115-138.

1. Vaccine comprising IL-18 or at least one fragment thereof and pharmaceutically acceptable adjuvants.

2. Vaccine according to claim 1, wherein at least one fragment of IL-18 in its original or multimerized form is coupled to a carrier molecule.

3. Vaccine according to claim 1, wherein the IL-18 is human, canine, feline or equine IL-18.

4. An IL-18 vaccine for use in medicine.

5. A process for the preparation of a vaccine according to claim 1 characterized by cloning of the cDNA, or a genomic sequence of IL-18 or a region encoding more than 5 amino acids thereof, ligating the same into a suitable vector, transforming the vector into an eukaryotic or prokaryotic host cell for the production of a fusion protein, containing the entire IL-18 sequence or a region thereof its original or in an mutated or multimerized form, and purifying and optionally mixing the obtained fusion protein with a suitable adjuvant.

6. The use of a fusion protein consisting of the entire or parts of IL-18 from the species to be treated and a foreign carrier protein for production of a vaccine for medical use.

7. Vaccine according to claim 2, wherein the IL-18 is human, canine, feline or equine IL-18.


SEQUENCE LISTING (4 sequences)

SEQ ID NO: 1 - DNA, 581 bp, Canine
atggctgctaacctaatagaagacaattgcatcaaccttgtgaaaatgaaatttgttaacaatacactgtactttaaagcggaaagtgatgaaggcctggaatcagattactttggcaagcttgaacctaaactctcaatcatacgaaatttgaacgaccaagtcctcttcgttaacgagggaaatcaacctgtatttgggatatgcccgattctgactgtacagataatgcaccccataccatatttatcatctatatgtataaagatagcctcactagaggtctggcagtaactatctctgtgaagtataagacaatgtctactctctcctgtaagaacaaaactatttcctttcagaaaatgagtcctccggatagtatcaatgatgaaggaaatgacatcatattctttcagagaagtgttccaggccatgatgataagatacaatttgagtcttcattgtacaaaggacactttctagcttgtaaaaaagagaacgatcttttcaaactcattttgaaagacaaggatgaaaatggggataaatccataatgttcactgttcaaaacaagagctag

SEQ ID NO: 2 - protein, 193 aa, Canine
MAANLIEDNCINLVKMKFVNNTLYFKAESDEGLESDYFGKLEPKLSIIRNLNDQVLFVNEGNQPVFEDMPDSDCTDNAPHTIFIIYMYKDSLTRGLAVTISVKYKTMSTLSCKNKTISFQKMSPPDSINDEGNDIIFFQRSVPGHDDKIQFESSLYKGHFLACKKENDLFKLILKDKDENGDKSIMFTVQNKS

SEQ ID NO: 3 - DNA, 1145 bp, Homo sapiens
attctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 4 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED